(19) United States
(12) Patent Application Publication
(10) Pub. No.: US 2024/0252731 A1
(43) Pub. Date: Aug. 1, 2024

(54) A Secondary Plasma Device for Collection of Cell-free Nucleic Acids from Blood Plasma During Extracorporeal Blood Processing by Clinical Apheresis Machines

(71) Applicant: MESA ALTA RESEARCH, LLC, Albuquerque, NM (US)

(72) Inventor: Mark Brenneman, Albuquerque, NM (US)

(73) Assignee: MESA ALTA RESEARCH, LLC, Albuquerque, NM (US)

(21) Appl. No.: 18/563,977

(22) PCT Filed: May 24, 2022
(86) PCT No.: PCT/US22/30761

§ 371 Date: Nov. 24, 2023

Related U.S. Application Data
(60) Provisional application No. 63/192,847, filed on May 25, 2021.

(51) Int. Cl. A61M 1/34; A61M 1/36; C12N 15/10

(52) U.S. Cl. CPC A61M 1/3496; A61M 1/3635; C12N 15/101

(57) ABSTRACT

A secondary plasma device (SPD) and methods for its use with apheresis machines or similar extracorporeal blood processing systems and capable of collecting cell-free nucleic acids (cfNA) directly from a subject's circulating plasma, the SPD comprising:
i. a sterile capsule having an inlet port and an outlet port and enclosing a filtration/adsorption medium, and configured to receive plasma from the extracorporeal blood processing circuit through the inlet port, such that plasma enters under pressure and flows through the filtration/adsorption medium then exits through the outlet port to a return path of the extracorporeal blood processing circuit leading back to the subject; and
ii. the filtration/adsorption medium bearing fixed positive charge that captures cfNA from plasma by anion exchange, and from which cfNA can be subsequently recovered in quantities 10 to 1000-fold greater than the amount of cfNA contained in a 5 ml venipuncture specimen of the subject's blood.

### CROSS-REFERENCE TO RELATED APPLICATIONS

This application claims priority to and the benefit of the filing of U.S. Provisional Patent Application No. 63/192,847 entitled “A SECONDARY PLASMA DEVICE FOR COLLECTION OF CELL-FREE NUCLEIC ACIDS FROM BLOOD PLASMA DURING EXTRACORPOREAL BLOOD PROCESSING BY CLINICAL APHERESIS MACHINES”, filed on May 25, 2021, and the specification and claims thereof are incorporated herein by reference.

### BACKGROUND OF THE INVENTION

### Field of the Invention

The present invention relates to the collection and isolation of circulating cell-free nucleic acids from blood for use as biomarkers in liquid biopsy tests for the diagnosis and prognosis of disease states, particularly cancers.

### Background and Significance of the Technology

The term liquid biopsy refers to assessment of disease states through detection of specific biological molecules, or biomarkers, in blood or other body fluids that can be collected by minimally invasive means. This contrasts with surgical biopsies, in which samples of tissue are excised through inherently invasive procedures. Biomarkers may be protein, lipid, carbohydrate or nucleic acid molecules that become altered in characteristic, disease-specific ways. Alterations may be in the quantity of a biomarker molecule or in its chemical structure. The concept and technologies of liquid biopsy have emerged mainly in relation to cancer, but will likely find application in other forms of disease as well, such as metabolic and neurodegenerative disorders, autoimmunity, and cardiovascular disease.

Liquid biopsies based on detection of circulating DNA fragments shed into the blood from cancer cells, i.e., circulating tumor DNA (ctDNA), have great potential to improve clinical management of cancer patients. For patients with malignant solid tumors, liquid biopsies could provide a powerful complement to imaging studies. After primary surgery and tumor genotyping, liquid biopsy could be employed for sensitive, blood-based detection of occult residual disease or metastases, for iterative monitoring of responses to radiation or drug therapies, and in post-treatment surveillance for cancer recurrence. Liquid biopsies may find eventual utility in early detection of new cancers as well, particularly if used selectively for individuals with known genetic cancer predispositions or identified pre-cancerous conditions such as ductal carcinoma in-situ of the breast or prostatic hyperplasia.

However, liquid biopsies have not yet achieved wide translation into routine oncology practice. This has been largely due to difficulty in gathering enough reliable data from trials of liquid biopsy tests to clinically validate them and establish their utility in supporting treatment decisions. A 2018 position paper issued jointly by the College of American Pathologists (CAP) and the American Society of Clinical Oncologists (ASCO) summarized findings of an expert review panel, in part, as follows:
Despite the extremely high level of current enthusiasm, deployment of ctDNA assays in routine clinical practice requires evidence of clinical utility. There is little evidence of clinical validity and clinical utility to support wide-spread use of ctDNA assays in most patients with advanced cancer . . . .

One of the greatest obstacles to establishing clinical validity and utility cited by the CAP/ASCO review panel is the highly variable quality and quantity of cfDNA specimens recovered from venipuncture blood draws, and the impacts of this variability on sensitivity and accuracy of liquid biopsy tests:
Insufficient evidence exists to resolve major remaining questions regarding retrospective studies, . . . especially in terms of sensitivity of the assay for ctDNA . . . it is unknown whether patients who are considered negative are truly negative and whether serial values truly reflect increase or decrease of the biomarker.

A standard 5 ml blood draw is roughly 1/1000^{th} of an adult's total circulating blood volume. The yield of total cell-free DNA (cfDNA) recoverable from it by commercially available cfDNA extraction kits varies widely among individual subjects, but typically ranges from 6 to 36 ng (higher cfDNA recoveries are sometimes seen in cancer patients with advanced, rapidly growing tumors). This is about 1000 to 6000 diploid genome-equivalents, of which only a fraction (possibly a very small one), will be ctDNA as opposed to cfDNA from normal tissue cells. The ctDNA fraction of a total cfDNA specimen will be further diminished if the specimen is contaminated with normal cellular DNA released by lysis of leukocytes occurring after the blood is drawn. Contamination of this kind has been a major problem in studies, and has been linked to vagaries of post-collection handling of blood draw specimens, such as delayed or incomplete separation of plasma from blood cells. As a consequence of these limitations, an informative ctDNA biomarker may be represented by only a handful of intact molecules in total cfDNA recovered from a venipuncture blood draw specimen or, if its total occurrence in the blood circulation was less than a few thousand molecules at the time of blood draw, possibly none at all.

Quantities and quality of cfDNA specimens recovered from venipuncture blood draws are thus often marginal for detection of ctDNA biomarkers in cancer patients, even by the most sensitive methods available, i.e., digital PCR assays, and wholly inadequate as inputs for next-generation DNA sequencing with broad genomic coverage and high depth. Yields of total cfDNA, and hence ctDNA for liquid biopsy, could be increased by taking larger blood draws. However, blood volumes that can be safely removed from a debilitated cancer patient have limits, and acceptable blood draw volumes will provide only incremental gains in cfDNA yield, which may or may not usefully increase the sensitivity of a ctDNA liquid biopsy test. Moreover, larger blood draws alone cannot address problems of poor specimen quality associated with post-collection deterioration of blood-draw specimens.

Cell-free, circulating RNA (cfRNA) molecules have also attracted increasing interest as liquid biopsy biomarkers, because of the insights they potentially offer into altered patterns of gene expression associated with cancers, metabolic and cardiovascular disease, neuro-degenerative conditions and other disease syndromes. cfRNA species of interest include messenger RNAs (mRNA), long non-coding RNAs (lncRNA) and a variety of small non-coding RNAs, especially micro-RNAs (miRNA). A large portion of the total cfRNA present in circulating blood is now known to reside within membrane-bound extracellular vesicles (EVs) released from cells of all or nearly all tissues in both normal and pathogenic states. EVs have been classified according to size, contents and mechanisms of release from their cells of origin, and are differentiated as microsomes/microvesicles, exosomes, oncosomes, apoptotic bodies, etc. Exosomes in particular have come to the fore, both as a promising source of miRNAs and mRNAs that may reflect pathogenic states in their cell types of origin, and as potential vectors for delivery of therapeutic nucleic acid molecules into tissue cells. In addition to cfRNAs, distinct fractions of total cfDNA have also been found to reside within some EVs, including exosomes, apoptosomes and oncosomes. Although EVs are abundant in the circulation and can be readily isolated from venipuncture blood draws, the proportion of EVs carrying specific cfRNA and/or cfDNA molecules having diagnostic significance may be extremely small. Thus, improved capture of EVs, as well as the free-floating, non-vesicular fraction of cfRNA and cfDNA, is of great importance to the development of liquid biopsy technology.

Viral DNAs and RNAs are another class of cell-free nucleic acids potentially present in the circulation. Active viral infections can usually be detected using plasma volumes available by venipuncture blood draw. In some situations, however, circulating titers of virus particles (VPs) or virus-like particles (VLPs) may be too low to detect reliably in blood-draw specimens. This will be of particular concern in relation to the emerging significance of reactivated endogenous retroviruses and latent viral infections of other kinds. In the realm of gene therapy, extremely sensitive detection of transfused viral vectors will be important in monitoring their cellular uptake and clearance from the circulation.

Problems of low, sensitivity-limiting cfNA specimen quantity and quality can potentially be overcome by collecting cfNA through an apheresis procedure. Clinical apheresis machines draw blood from a peripheral or central venous catheter, transiently separate the formed elements (blood cells and platelets) from plasma to allow removal or exchange of chosen components, and then return the processed blood to the patient. Apheresis is also used routinely in blood banking to collect plasma and platelet donations. In a typical therapeutic plasma exchange (TPE) procedure, an apheresis machine will process 1.5 to 2 times the total circulating blood volume. Blood being drawn is progressively admixed with blood being returned, so that the entire circulation of a patient is never completely processed, but a TPE procedure effectively processes about 50% to 75% of the circulating plasma volume in about two to three hours [3]. For an adult, this will be 2 to 3 liters of plasma, i.e., 800 to 1200 times the ˜2.5 ml of plasma obtained from a 5 ml blood draw. Thus, if cfDNA is efficiently collected through real-time processing of plasma during an apheresis procedure, the yields of cfDNA available for liquid biopsy tests can be increased in similar proportion, e.g., into a range of about 4 to 40 μg. Contamination of recovered ctDNA with normal cellular DNA from lysis of leukocytes is also reduced, because cfDNA is sequestered from blood cells continuously during the process of collection, not in a later, post-collection step after some leukocyte lysis may have occurred.

Collection of cfDNA and/or cfRNA from plasma during apheresis can be accomplished using a secondary plasma device (SPD), i.e., a special filter inserted into the extracorporeal blood circuit of a clinical apheresis machine, between the plasma pump and the plasma collection or return bag (FIG. 2). In the past, apheresis SPDs have been created for therapeutic purposes, i.e., to remove pathogenic substances from the bloodstream, usually through semi-selective adsorption. Examples include SPDs for removal of excessive high-density lipoprotein complexes in treatment of familial hypercholesterolemia, and SPDs for reduction of immunoglobulins in autoimmune disorders including lupus erythematosus. The present invention is a departure from the historically therapeutic intent of clinical apheresis, in that the disclosed SPD is used in apheresis procedures performed for purely diagnostic purposes; to collect and concentrate biomarker molecules that occur at extremely low levels in the blood circulation.

Technical requirements and desirable features of an SPD for collecting cell-free nucleic acids during apheresis include the following:
i. High binding affinity for cfDNA and cfRNA, whether present in free-floating, non-vesicular forms or within an EV/VP compartment. The filtration/adsorption medium used must be able to efficiently bind cfDNA/RNA and EVs/VPs present in plasma under physiological conditions and retain them through post-collection purification washes, yet allow their complete elution for recovery and concentration afterward.
ii. High ratio of filtration/adsorption surface area to internal device volume. The surface area available for binding of nucleic acids by anion exchange should be large enough to bind the maximum load of cfDNA and/or cfRNA projected to be present in the circulation. High filtration surface area is also necessary to reduce the likelihood of clogging due to surface loading with plasma macromolecular complexes or colloidal particles. Internal volume of the device should be as low as possible because an SPD should add as little as possible to the total volume of the extracorporeal blood circuit for safety reasons. Low internal device volume will also reduce the volume of buffer needed to elute captured cfDNA/RNA from the adsorption medium and hence the eluate volume from which they must be further concentrated.
iii. High flow rates at low pressures. Plasma flow rates through the SPD must be high enough at maximum pressures attainable in an apheresis system (500-600 mmHg) that volumes up to six to eight liters can pass through it during a procedure of reasonable time, e.g., 2 hours or less. This will require sustained average flow rates of at least 50 ml/min. The filtration/adsorption medium used must therefore have a low intrinsic flow resistance and resist fouling and occlusion by plasma components including lipids, proteins or lipoproteins and other substances in the forms of macromolecular complexes or colloidal particles.
iv. Biocompatibility. Because plasma passing through the SPD will usually be returned to the patient during the apheresis cfDNA/RNA collection procedure, the adsorption medium and all other materials present in the device must be well characterized as fully biocompatible, i.e., previously FDA-approved or amenable to regulatory approvals for use in blood-contact medical devices.

### BRIEF SUMMARY OF THE INVENTION

It is an object of the invention to provide a secondary plasma device (SPD), compatible with existing clinical apheresis machines or similar systems for extracorporeal blood processing, that can adsorb cell-free DNA and/or RNA molecules from a large fraction of the total circulating plasma volume and sequester them for subsequent recovery, by exploiting the principles of anion exchange. It is a further object of the invention to provide methods for (i) using said SPD to collect cell-free DNA and/or RNA from the circulation of a subject during an apheresis procedure; (ii) stabilization of captured cfNA after collection to preserve them during transport of the SPD from clinic to laboratory, and (iii) laboratory processing of the SPD to recover the captured DNA and/or RNA in states of purity and concentration suitable for input to molecular analytical technologies such as polymerase chain reaction (PCR) assays and DNA sequencing. The SPD and methods disclosed herein recover cfDNAs and cfRNAs, including those contained within extracellular vesicles or viral particles, from the blood circulation in a state of preservation suitable for analysis by molecular techniques such as digital polymerase chain reaction assays and next-generation sequencing of DNA and RNA. The disclosed SPD and methods recover cfDNA and cfRNA in amounts up to 1000 times greater than available from venipuncture blood-draw specimens, sufficient to allow repeated tests or complimentary modes of analysis from a single specimen, as well as banking of surplus cfDNA/cfRNA for retrospective comparisons and correlative research studies.

### BRIEF DESCRIPTION OF THE FIGURES

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 illustrates capture of cell-free nucleic acids (cfNA) from circulating blood plasma by anion exchange, in which the plasma passes through a filtration/adsorption medium bearing a high density of positively charged groups according to one embodiment of the present invention.

FIG. 2 illustrates integration of a secondary plasma device (SPD) for capture of cell-free nucleic acids (cfNA) into the extracorporeal blood circuit of a clinical apheresis system according to one embodiment of the present invention.

FIG. 3 illustrates a secondary plasma device (SPD) capsule for capture of cell-free nucleic acids (cfNA) from separated plasma, containing a positively-charged filtration/adsorption medium in the form of hollow-fiber membranes and configured for transmembrane (“dead-end”) filtration, according to one embodiment of the present invention.

FIG. 4 illustrates a secondary plasma device (SPD) capsule for capture of cell-free nucleic acids (cfNA) from separated plasma, containing a positively-charged filtration/adsorption medium in the form of hollow-fiber membranes and configured for tangential (“cross-flow”) filtration, according to one embodiment of the present invention.

FIG. 5 illustrates methods for post-collection handling of the SPD to stabilize captured nucleic acids during transport from clinic to laboratory, and for laboratory processing of the SPD to recover captured nucleic acids in purified and concentrated form suitable for downstream analysis according to one embodiment of the present invention.

FIG. 6 illustrates methods for post-collection handling of the SPD to stabilize captured extracellular vesicles (EVs), virus particles (VPs) or virus-like particles (VLPs) during transport from clinic to laboratory, and for laboratory processing of the SPD to recover captured EVs, VPs or VLPs in purified and concentrated form suitable for downstream analysis according to one embodiment of the present invention.

### DETAILED DESCRIPTION OF THE INVENTION

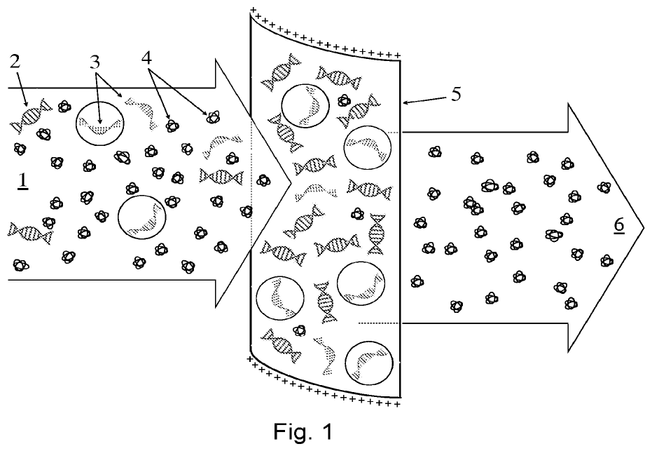

It is an object of the invention to provide an auxiliary filter, or secondary plasma device (SPD), that can be used with existing systems for extracorporeal blood processing such as clinical apheresis machines, to selectively adsorb cell-free nucleic acids (cfNA), comprising cell-free DNA (cfDNA) and/or cell-free RNA (cfRNA) from a large fraction of a human subject's total circulating plasma volume and sequester them for subsequent recovery. The SPD contains a polymeric filtration/adsorption medium that displays a high density of fixed positive charges on its surface at physiological pH and is thus able to capture negatively charged nucleic acid molecules, or supramolecular complexes that contain them, in plasma flowing though it by the mechanism of anion exchange (FIG. 1). It is a further object of the invention to provide methods for (i) using said SPD to collect cfNA from the circulation of a subject during an apheresis procedure; (ii) stabilization of captured cfNA after the collection procedure to preserve them during transport of the SPD from the apheresis clinic to a central laboratory, and (iii) laboratory processing of the SPD to recover the captured cfNA in a state of high purity and concentration suitable for input to molecular analytical technologies such as polymerase chain reaction (PCR) assays and DNA sequencing.

For purposes of this description, the term cell-free nucleic acids (cfNA) will include all DNA and RNA present in the blood circulation but not contained within intact cells. This will specifically include DNA and RNA contained within membrane-bound extracellular vesicles (EVs), in virus particles (VPs) or in virus-like particles (VLPs), most forms of which are also amenable to capture on anion exchange media owing to their high levels of negative surface charge. Moreover, the described SPD can also be used to collect intact EVs, VPs or VLPs and recover them as such through modified methods. Circulating tumor cells (CTC), i.e., rare cells that have detached from a solid tumor and entered the bloodstream, are another form of cancer biomarker that has attracted much interest. It should be noted, however, that CTCs will not be collected by the SPD and methods described herein, because the SPD is exposed only to plasma that has been separated from all cells in the bloodstream during apheresis.

Referring now to FIG. 1, capture of cell-free nucleic acids (cfNA), comprising extracellular molecules of DNA and/or RNA, directly from circulating blood plasma by anion exchange, in which the plasma is passed through a filtration/adsorption medium bearing a high density of positively charged groups, is illustrated according to one embodiment of the present invention. Input stream of plasma (1) that has been separated via apheresis from all blood cells (including circulating tumor cells and other non-diseased cells) and platelets, but rich in plasma lipids, proteins and cfNA. Cell-free nucleic acids including both DNA and RNA, may be present as free molecules, in complex with proteins, or contained within membrane-bound extracellular vesicles, virus particles or virus-like particles. Within the plasma input are molecules of small, cell-free DNA (2), molecules of cell-free RNA (3), which may be enclosed within extracellular vesicles or not and molecules of serum protein (4). Filtration face of an anion-exchange membrane (5) which, under physiological conditions of pH and ionic strength, will retain negatively charged solutes and colloidal particles including free and protein-bound cfNA and some plasma proteins such as serum albumens. cfNA are retained in preference to serum proteins due to higher density of negative charge. Membrane-bound extracellular vesicles, and enveloped virus particles or virus-like particles if present, are also retained on the anion-exchange matrix due to a high density of negative surface charge on their membranes. Colloidal lipid complexes may be retained to greater or lesser extent, depending upon pore size and structure of the filtration/adsorption medium. Output stream of filtered plasma (6), depleted of cfNA and EVs, VPs or VLPs, but still carrying the bulk of plasma proteins and lipids. In one embodiment, output stream (6) is in fluid communication with the closed loop apheresis tubing as is the input stream (1).

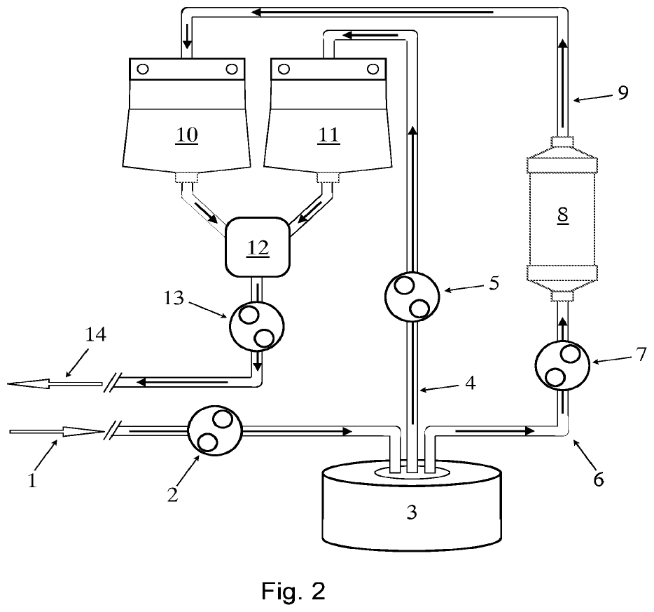

In a first aspect of the invention, the secondary plasma device (SPD) is a sealed, sterile capsule that can be integrated to the extracorporeal blood circuit of an apheresis system by connecting the SPD into an apheresis procedure tubing set at a point in the apheresis system where the SPD receives the output of the plasma pump and discharges to the plasma collection reservoir or treated plasma bag (FIG. 2). The plasma pump of an apheresis machine propels a stream of plasma that has just been separated from formed elements (red blood cells, white blood cells, circulating tumor cells, tissue cells, and platelets) by zonal centrifugation or alternately by filtration. This stream of plasma passes into an inlet port of the SPD, through the SPD and exits through an outlet port of the SPD to a treated plasma reservoir, where plasma is held until it is recombined with blood cells and returned to the patient (FIG. 2) or not returned to the patient depending on the reason for the apheresis treatment.

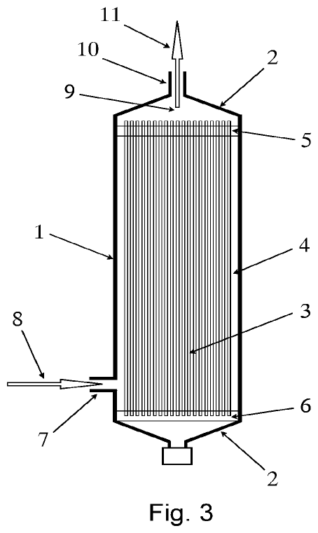

Referring now to FIG. 2, placement of a secondary plasma device (SPD) for capture of cell-free nucleic acids (cfNA) in the extracorporeal blood circuit of a clinical apheresis system is illustrated according to one embodiment of the present invention wherein blood intake from patient (1) flows to the blood intake pump (2). A hemoseparator (3), centrifugal or filtration type is positioned in the circuit. The cells and platelets (4) in concentrated suspension are separated and routed to a cell suspension line pump (5). The separated plasma (6) that is cfNA-rich is sent to a plasma line pump (7). A secondary plasma device (SPD) (8), (e.g., as illustrated in FIG. 3 according to one embodiment of the SPD) is in fluid communication with the separated plasma, which is depleted of cfNA after passing through the SPD and exits via the SPD outlet (9). A treated plasma bag (10) receives the cfNA depleted plasma and the cell concentrate bag (11) receives the cells and platelets (4) and these are combined as appropriate in the return reservoir (12), where cells and treated plasma and/or saline diluent are recombined and blood return pump (13) assists in returning the blood 14) to the treated patient.

In one embodiment, the SPD capsule contains a polymeric filtration/adsorption medium with a high density of fixed positive charges on its surface at physiological pH. It is thus able to capture nucleic acid molecules, which bear a high density of negatively charged phosphate groups, by the mechanism of anion exchange (FIG. 1). Said polymeric medium may take the form of a packed bed of resin beads, or a cast monolithic element with mesoporous structure, or a microporous filtration membrane. Positively charged chemical moieties on the medium may take the form of secondary, tertiary or quaternary amine groups, imine groups or others. In a preferred embodiment, the medium is a microfiltration membrane composed mainly of polysulfone (PS) or polyethersulfone (PES) that is modified to carry positively charged quaternary amine groups. The quaternary amine groups may have been incorporated into PS or PES membranes either in the course of their manufacture or as a post-manufacture treatment of uncharged membranes by methods, for example, such as those described by Wang and Zepf (U.S. Pat. No. 6,565,748).

Pores in the SPD filtration/adsorption medium are sufficiently large that macromolecular solutes and supramolecular complexes present in plasma, such as proteins or protein complexes, lipids or lipoprotein complexes and small molecules of DNA or RNA, are not excluded from passage on the basis of size. Rather, they are either retained on the medium or pass through it mainly on the basis of charge, with negatively charged solutes including nucleic acids tending to be retained, and neutral or positively charged solutes passing through. Accordingly, pores in the medium are generally 100 nm (0.10 microns) or greater in mean diameter, with pores of 450 nm (0.45 microns) or greater being preferred.

Where microfiltration membranes are used as the filtration/adsorption medium of the SPD, they may have a symmetric or asymmetric membrane structure. Symmetric membranes have an essentially uniform structure in cross section, with pores being consistent in size through the entire membrane thickness, and the two sides of the membrane having similar microscopic surface textures. In contrast, asymmetric membranes are fabricated by methods that result in an open, mesh-like microscopic structure on one side and a smooth, skin-like surface with pore openings of defined size on the other side. Membranes with asymmetric structure can be advantageous in filtration of fluids like blood plasma that carry colloidal particles, because the open, mesh-like face of the membrane has some capacity to entrap colloidal particles as they enter and so reduce occlusion of the recessed, smaller pores. In a preferred embodiment, the filtration/adsorption medium of the SPD is a positively charged microfiltration membrane of asymmetric pore structure, with an open, mesh-like structure on the upstream side and a smooth surface with pores of defined size on the downstream side of the membrane.

In an alternative preferred embodiment, the filtration/adsorption medium is a hydrophilic but uncharged membrane of asymmetric structure, used in combination with positively charged nanoparticles. Nanoparticles are loaded onto the upstream (inlet) side of the membrane, and are small enough that they can enter the mesh-like surface of the membrane filtration face and be retained there, but large enough that they cannot pass through the defined pores at the downstream, skin-like side of the membrane. For example, monodisperse nanoparticles having a diameter of 200 nm can be used with an asymmetric membrane having a nominal 100 nm pore size. Suitable nanoparticles may be composed of an organic polymer such as polystyrene, or an inorganic matrix such as boron nitride, functionalized with positively charges groups such as quaternary amines. Alternately, nanoparticles may be composed of materials that inherently display a high density of positively charged groups at physiological pH, such as polyenthyleneimine or chitosan.

Hollow-fiber filtration membranes are widely used in extracorporeal blood-processing systems for dialysis, diafiltration, hemofiltration and hemoconcentration, in order to achieve extremely high ratios of filtration surface area to device volume. In a preferred embodiment of the SPD for collection of cell-free nucleic acids, the filtration/adsorption medium is a bundle of hollow-fiber, asymmetric PS or PES membranes having an open, mesh-like structure on the outer (extra-luminal) side of the membrane and a smooth, skin-like surface with pores of defined size on the inner (luminal) side of the membrane. In one embodiment, the surfaces of the PS or PES hollow-fiber membranes are modified to display a high density of fixed quaternary amine groups. The bundle of positively-charged hollow-fiber membranes is enclosed within a cylindrical capsule fitted with inlet and outlet ports and internal manifolds that direct the flow of plasma though the device.

Plasma flow through the SPD capsule and filtration/adsorption membranes therein may take the form of transmembrane or “dead-end” filtration, in which fluid may pass only through the pores of the membrane. Alternately it may be tangential or “cross-flow” filtration, in which fluid is free to move both across the surface and through the pores of the filtration membrane.

In a transmembrane embodiment, a bundle of asymmetric, hollow-fiber membranes is configured for dead-end filtration by enclosing them within a cylindrical casing, with the fiber lumens closed off at one end of the bundle and opening at the other end to an outlet manifold and outlet port (FIG. 3). Alternately, hollow fiber membranes may be configured as loops, with both luminal ends opening to the outlet manifold and outlet port. In either case, plasma enters the SPD through an inlet port opening to the extra-luminal space of the capsule (i.e., “shell feed”), where it meets the external surfaces of the hollow-fiber anion-exchange membranes and passes through them into the lumens. The plasma (now a filtrate) then flows through the lumens to a collecting outlet manifold and exits via an outlet port. In this manner, the entire volume of cell-free plasma produced by the apheresis system over the course of an cfDNA/RNA collection procedure passes through the pores of the hollow-fiber membranes of the SPD and exits as a filtrate.

Referring now to FIG. 3, a secondary plasma device (SPD) for capture of cell-free nucleic acids (cfNA) from separated plasma in the extracorporeal blood circuit of an apheresis system, according to one transmembrane embodiment of the present invention, is illustrated. Cylindrical plastic casing (1) with molded plastic end caps (2) provide a casing/housing for bundle of hollow-fiber filtration/adsorption membranes with positive surface charge for anion exchange (3). Extra-luminal space (4), internal to casing but external to hollow fibers, is between potting material (5), that seals off open ends of hollow fibers from extra-luminal space and potting material (6) that closes lower ends of hollow fibers. Inlet port (7) permits fluid communication with opening to extra-luminal space. Inward flow of cfNA-rich plasma (8) to extra-luminal space via inlet (7) permits fluid to pass through SPD to outlet manifold (9) via open luminal spaces of hollow fiber membranes (3). The fluid exits the SPD via outlet port (10) and the outward flow of filtrate (11), i.e., filtered, cfNA-depleted plasma is in fluid communication with the apheresis tubing to which the outlet is connected.

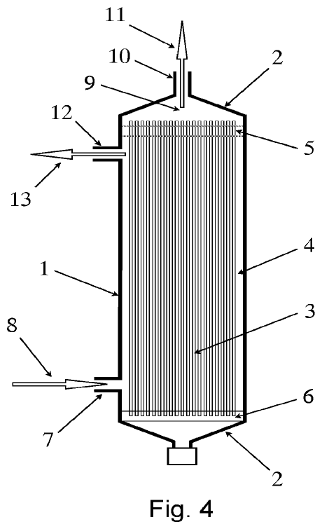

In another tangential filtration embodiment, hollow fiber membranes are configured just as described above for transmembrane filtration, but within a capsule that has an additional outlet port opening from the extra-luminal space (FIG. 4). In this embodiment, plasma enters the extra-luminal space of the SPD through an inlet port at the proximal end of the capsule, but flow then divides between two possible paths: plasma may flow tangentially along the outer surfaces of the hollow-fiber membranes and then exit the extra-luminal space as a retentate via an outlet port at the distal end of the capsule, or it may pass through pores of the hollow fiber membranes and flow through their lumens to reach the luminal outlet manifold and outlet port as a filtrate. Plasma retentate and filtrate from the two outlet ports are combined and flow to the treated plasma bag for return to the patient.

Referring now to FIG. 4, a secondary plasma device (SPD) for capture of cell-free nucleic acids (cfNA) from separated plasma in the extracorporeal blood circuit of an apheresis system, according to one tangential embodiment of the present invention, is illustrated with the following elements: (1) cylindrical plastic casing; (2) molded plastic end caps; (3) bundle of hollow-fiber filtration/adsorption membranes with positive surface charge for anion exchange; (4) extra-luminal space, internal to casing but external to hollow fibers; (5) potting material, sealing off open ends of hollow fibers from extra-luminal space; (6) potting material, closing lower ends of hollow fibers; (7) inlet port, opening to extra-luminal space; (8) inward flow of cfNA-rich plasma to extra-luminal space; (9) outlet manifold, open to luminal spaces of hollow fiber membranes; (10) filtrate outlet port; (11) outward flow of filtrate, i.e., filtered, cfNA-depleted plasma; (12) retentate outlet port; (13) outward flow of retentate.

Dead-end and cross-flow embodiments of the SPD have operational trade-offs. In a dead-end filtration embodiment, all cell-free nucleic acid molecules necessarily come into very close proximity to the positively charged surface of the adsorption/filtration membrane, as all plasma must pass through its pores. Dead-end filtration may thus offer the most effective capture of cell-free nucleic acids, but may be prone to clogging of membrane pores by colloidal matter present in plasma. Cross-flow filtration is generally more resistant to clogging than dead-end, because tangential flow across the filtration face tends to sweep colloidal particles away from membrane pores. However, in cross-flow embodiments of the SPD, it is possible that some fraction of nucleic acid molecules may pass through in the retentate flow and avoid capture on the membrane surface, thus reducing the efficiency of capture and potential recovery. This effect can be reduced by constructing the SPD for minimized extra-luminal volume.

In a second aspect of the invention, methods are disclosed for use of the SPD in apheresis procedures for cfDNA/RNA collection, for post-collection handling of the SPD to maximize preservation of the captured nucleic acids it contains, and for subsequent laboratory processing of the SPD to recover the captured nucleic acids in a purified and concentrated state.

To collect circulating, cell-free nucleic acids from the bloodstream of a patient, the SPD is aseptically connected into a sterile, single-use apheresis procedure tubing set via Luer-lok connectors. The SPD can be used with apheresis machines that separate the formed blood elements from plasma by zonal centrifugation or by hemofiltration. In either case, the SPD is inserted into the procedure tubing set at a point beyond that where plasma is separated from blood cells and platelets, i.e., between the plasma pump and the plasma collection reservoir or treated plasma bag (FIG. 2). In this way, the separated plasma is directed into the SPD, where molecules of cell-free nucleic acids will be retained on the anion-exchange filtration/adsorption medium, while the bulk of plasma volume and solutes pass through it for ultimate return to the patient's bloodstream. In a preferred application, the procedure tubing set used is one designed for therapeutic plasma exchange and/or RBC exchange procedures, in which a shunt line with Luer-lok connectors is provided, suitable for inserting a secondary plasma device at the correct position.

One or more embodiments of the disclosed SPD are intended primarily for use with clinical apheresis systems, but could conceivably be deployed, with some modifications, in other kinds of extracorporeal blood processing systems including but not limited to dedicated plasmapheresis machines (used to collect plasma donations), renal dialysis machines, or heart-lung machines. Dedicated plasmapheresis machines are simplified versions of clinical apheresis machines and have the required capabilities to produce a stream of plasma separated from formed elements and to return the formed elements (plus diluent) to the donor. Their use in a cfNA collection procedure would require mainly that the tubing set be modified to direct plasma into the disclosed SPD and connect its outlet port to the blood return stream. In the case of renal dialysis machines, the dialysis cartridge would be replaced with the disclosed SPD positioned downstream of a hemo-separator capsule (similar to those used by some clinical apheresis machines to separate plasma from formed elements). An analogous arrangement of hemo-separator plus SPD would be possible in conjunction with cardiopulmonary bypass or extracorporeal membrane oxygenation machines.

Before beginning cfNA collection, several device-volumes of sterile physiological saline that includes the chosen anticoagulant agent (citrate or heparin) are passed through the SPD to equilibrate the adsorption medium and ensure all air has been purged from the device and associated tubing. A device-volume here means the total fluid volume contained within the SPD when completely filled. Inlet pressure to the SPD (pressure between the plasma pump and the SPD) should be monitored during cfNA collection, so that it will be apparent if the SPD is becoming clogged by colloidal matter. Plasma flow rates through the SPD may range from 10 ml/min to 100 ml/min or more, depending upon many factors including the model of apheresis machine used and its chosen settings or procedure programming, and the load of colloidal material present in an individual patient's blood. Anticoagulation should be with citrate (ACD-A) rather than heparin if possible, as heparin has a high negative charge density and may compete to some extent with nucleic acids for binding to the anion-exchange filtration/adsorption medium. The apheresis procedure for cfNA collection will be run much as for a typical therapeutic plasma exchange, with the important distinction that all plasma processed may be returned to the patient rather than being replaced. Although therapeutic plasma exchange procedures may extend for two to three hours, a shorter apheresis time with the SPD, e.g., one hour or less, may suffice to collect enough cfDNA/cfRNA for all anticipated analyses.

At the end of the collection procedure, nucleic acids captured in the SPD are stabilized to protect them from degradation during transport of the SPD from the apheresis clinic to a qualified processing laboratory. This is accomplished by immediately disconnecting the SPD from the extracorporeal blood circuit and flushing it with several device-volumes of a preservative buffer provided to the apheresis clinic with the SPD. Preservative buffer is manually injected with a syringe through the inlet port of the SPD, with displaced volume discharged from the outlet port into a suitable medical waste container. Then, with the SPD completely filled by preservative buffer, the inlet and outlet ports are sealed with watertight caps that will remain securely in place during transport to a designated laboratory (for processing of the SPD to recover the captured cfNA).

The preservative buffer used to stabilize and protect nucleic acids in the SPD includes chemical agents such as citrate or ethylenediamine tetraacetic acid (EDTA) that chelate divalent cations, notably calcium and magnesium, at concentrations sufficient to prevent formation of fibrin aggregates and to inhibit activity of any nucleolytic enzymes retained from plasma that might otherwise degrade the cell-free DNA/RNA (e.g., 10 mM EDTA). It also includes one or more non-ionic or zwitterionic surfactants, such as Triton-X 100 or CHAPS, at concentrations sufficient to solubilize any plasma lipids or lipoprotein complexes retained by the SPD (e.g., 0.5% Triton X-100). The preservative buffer is formulated to ionic strength and pH that will ensure all cell-free nucleic acids captured by the SPD remain bound on the anion-exchange filtration/adsorption medium, e.g., sodium chloride at a concentration of 300 mM or less and pH buffered to 7.4 to 8.0.

Optionally, the preservative buffer can also be formulated to begin the first step in DNA/RNA purification during transport from apheresis clinic to processing lab. This step is enzymatic digestion and solubilization of proteins present in the specimen, which are regarded as contaminants of the cell-free DNA/RNA for purposes of downstream analysis. To this end, the preservative buffer for the SPD may also include one or more proteolytic enzymes, such as proteinase K, that will digest plasma proteins, including nucleases present in plasma and particularly serum albumins, which may be retained on the filtration/adsorption medium of the SPD in significant amounts owing to their abundance and negative charge.

One embodiment of post-collection laboratory processing of the SPD to recover captured cell-free nucleic acids in purified and concentrated form comprises several discrete steps: (i) a preliminary proteolytic digestion, if this has not been performed during transport to the laboratory; (ii) washing to remove digested or partially digested proteins, solubilized lipids and other contaminating plasma substances; (iii) elution of bound nucleic acids from the anion-exchange filtration/adsorption medium with a small volume of high-salt elution buffer, followed by (iv) desalting and further concentration of the eluted nucleic acids for downstream analysis. These methods are a departure from those typical of commercial kits for extraction of cell-free nucleic acids from blood. Protocols for currently available commercial kits generally comprise a series of processing steps that begin with whole blood or plasma separated from whole blood obtained by venipuncture blood draw, followed by capture of nucleic acids on an adsorption medium (usually a silica matrix) and further processing thereupon, and finally an elution of purified DNA/RNA in small volume. Here, in contrast, all steps in purification of cfDNA/RNA except desalting occur within the SPD, subsequent to their direct capture from separated plasma during the apheresis collection procedure.

In one embodiment, washing steps entail passing relatively large volumes of one or more aqueous wash solutions through the SPD by means of a syringe or, preferably, a peristaltic pump. Wash volumes are typically 10-100 device-volumes. For example, in an SPD having a total internal volume of 5 ml, wash volumes may range from 50 ml to 500 ml. Wash solutions are pH-buffered to neutral or mildly basic pH (i.e., pH 7.0 to pH 8.0) and contain salts (NaCl and/or others) at moderate concentrations that will displace biomolecules with weak negative charge (such as serum albumins) from the anion-exchange filtration/adsorption medium, yet not displace captured cell-free nucleic acid molecules (e.g., 300 mM to 800 mM NaCl). Wash buffers may also contain one or more non-ionic or zwitterionic surfactants such as Triton X-100 or CHAPS at concentrations sufficient to solubilize lipids and lipoproteins and thus aid their removal from the anion-exchange filtration/adsorption medium (e.g., 0.5% Triton X-100). Wash solutions may also include an alcohol or glycol, such as ethanol, isopropanol, glycerol, polyethylene glycol or others, to reduce solubility of nucleic acids and thereby aid their retention on the anion-exchange filtration/adsorption medium (e.g., 30% to 50% ethanol).

In one embodiment, elution of captured cell-free nucleic acids from the SPD in purified and concentrated form is accomplished by passing relatively small volumes of an aqueous, high-salt elution buffer through the SPD by means of a syringe or pump. Elution volume is typically 2 to 10 device-volumes, e.g., 10 ml to 50 ml for an SPD having total internal volume of 5 ml. The elution buffer is adjusted to mildly basic pH (i.e., pH 7.4 to pH 8.0) and contains salt (NaCl and/or others) at a concentration high enough to displace bound nucleic acids from the anion-exchange filtration/adsorption medium (e.g., 1.25 M to 2 M NaCl) so that they may be recovered from eluate exiting the SPD. Eluate may be collected as a single volume or as successive fractions that contain variable amounts of eluted DNA and/or RNA.

In one embodiment, cfDNA/RNA recovered in the eluate may then be further concentrated, and salts removed, using any of several standard laboratory means such as:
i. precipitation of eluted cfNA with alcohol, followed by washing the cfNA pellet to remove salts and redissolving it in a small volume, e.g., 100-500 μl, of water or a low-salt buffer such as standard Tris-EDTA (TE) buffer;
ii. adsorption of eluted ctNA to the surface of silica-coated paramagnetic beads or a silica-coated filtration membrane in the presence of guanidinium salts, followed by washing to remove salts and elution in a small volume of water or a low-salt buffer such as standard TE buffer;
iii. washing and concentration of cfNA with a centrifugal ultrafiltration device having a filtration membrane with appropriate molecular-weight cut-off, e.g., 3,000 to 10,000 daltons.

Yields of total cell-free nucleic acids recovered from the SPD will vary widely among human subjects undergoing the apheresis collection procedure and will depend upon the duration chosen for the collection procedure, i.e., the volume of plasma processed. For a maximum collection period of 2 to 3 hours, in which 50% to 75% of the circulating plasma volume will pass through the SPD, a total yield of cell-free nucleic acids recovered by the methods outlined here may range up to 30 μg or more.

If downstream analytical techniques require cfDNA without the presence of cfRNA, this may be obtained by treating a portion of the recovered total cell-free nucleic acids with an RNA-specific nuclease (RNase). Similarly, cfRNA without cfDNA may be obtained by treating a portion of the recovered total cell-free nucleic acids with a DNA-specific nuclease (DNase). Digestions with RNase or DNase should be followed by a complete buffer exchange using, for example, microfuge-format spin filters with an appropriate molecular-weight cut-off, e.g., 3,000 to 10,000 daltons.

In one aspect, cell-free DNA recovered from the SPD by these methods will be sufficiently pure to serve as direct inputs for analytical techniques such as digital polymerase chain reaction (dPCR) and next-generation sequencing (NGS). Cell-free RNA recovered by this process will be of sufficient purity to serve as direct input for reverse transcription reactions followed by dPCR or NGS.

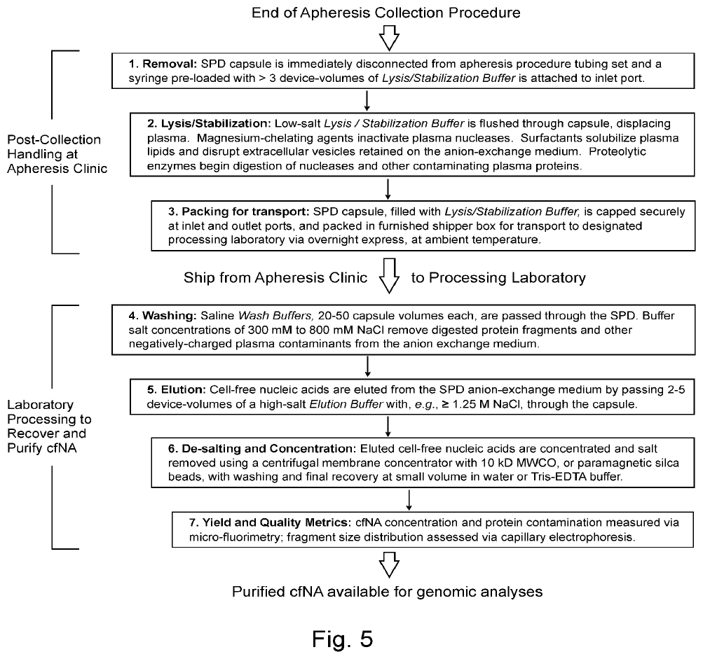

Referring now to FIG. 5, an overview of methods for post-collection handling of the SPD to stabilize and preserve captured cell-free nucleic acids, and for subsequent laboratory processing to recover cell-free nucleic acids in purified and concentrated form suitable for downstream analysis is illustrated according to one embodiment of the present invention with reference to the following steps: (1) removal from the apheresis procedure tubing set; (2) lysis and stabilization; (3) packing for transport; (4) washing to remove impurities; (5) elution of cfNA; (6) de-salting and concentration; (7) yield and quality metrics. The steps produce a cfNA that is isolated from the cfNA-rich plasma and the cfNA isolated from the plasma at the SPD can be sent to a diagnostic genomics lab for further analysis.

For analysis of biomarker molecules known or thought to be concentrated in the extracellular vesicle (EV) compartment, one embodiment provides for harvesting intact EVs separately from the non-vesicular fraction of total cfNA. This may apply particularly to certain RNA molecules concentrated within EVs, but also to DNA fragments and proteins with potential utility as biomarkers. Similarly, it may be of interest to collect from the circulation intact virus particles (VPs) or recombinant virus-like particles (VLPs) such as lentiviral, adenoviral or AAV-derived vectors used to deliver gene therapy or nucleic acid vaccines, in order to monitor their cellular uptake and clearance from the circulation.

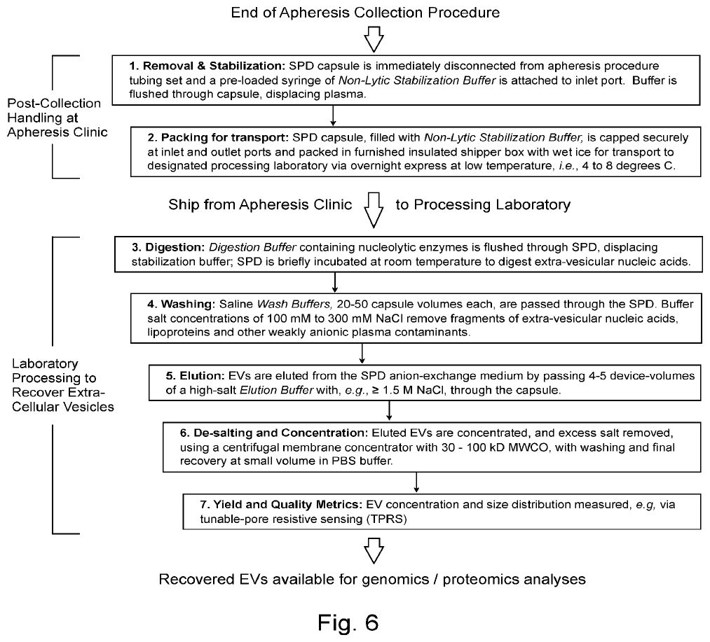

One embodiment of the SPD can be used to capture and recover EVs and VPs or VLPs from the blood circulation, because of the relatively high negative surface charges that characterize such particles. Further, the claimed SPD can be used to collect EVs from the circulation of a subject on a preparative scale, for subsequent use as delivery vectors for drugs or therapeutic macromolecules including DNAs, RNAs, proteins or others. However, to recover EVs or VPs or VLPs in an intact state and separately from non-vesicular, cell-free nucleic acids requires alternative methods for post-collection handling and laboratory processing of the SPD, which are summarized in FIG. 6.

The apheresis collection procedure for capture of EVs, VPs or VLPs is similar to that used for collection of total cell-free nucleic acids, but differs in several respects. At the end of the collection procedure, the SPD is immediately disconnected from the procedure tubing set and flushed with several device-volumes of an alternative preservative buffer that does not include surfactants or detergents, which may tend to compromise vesicular membranes or viral envelopes. Neither does the preservative buffer include proteolytic enzymes, which might degrade protein markers displayed on vesicular membranes or viral envelopes. Preservative buffer is manually injected with a syringe through the inlet port of the SPD to displace all remaining plasma, with displaced volume discharged from the outlet port collected into a suitable medical waste container. Then, with the device completely filled by preservative buffer, the inlet and outlet ports are sealed with watertight caps that will remain securely in place during transport.

The preservative buffer used to stabilize and protect EVs, VPs or VLPs in the SPD includes chemical agents such as citrate or ethylenediamine tetraacetic acid (EDTA) that chelate divalent cations, notably calcium and magnesium, at concentrations sufficient to prevent formation of fibrin aggregates and to inhibit activity of any nucleolytic enzymes retained from plasma (e.g., 10 mM EDTA). It may also include protease inhibitors and/or RNase inhibitors. The preservative buffer is formulated to ionic strength and pH that will ensure EVs, VPs or VLPs captured by the SPD remain bound on the anion-exchange filtration/adsorption medium, e.g., sodium chloride at a concentration of 300 mM or less and pH buffered to 7.4 to 8.0.

Post-collection laboratory processing of the SPD to recover captured EVs, VPs or VLPs in partially purified and concentrated form in one embodiment of the present invention comprises at least three discrete steps: (i) washing to remove plasma proteins, lipoproteins and other contaminating plasma substances; (ii) elution of bound EVs, VPs or VLPs from the anion-exchange filtration/adsorption medium with a small volume of high-salt elution buffer, followed by (iv) desalting and further concentration of the eluted EVs, VPs or VLPs for downstream analysis.

Washing steps entail passing relatively large volumes of one or more aqueous wash solutions through the SPD by means of a syringe or, preferably, a peristaltic pump. Wash volumes are typically 10 to 100 device-volumes. For example, in an SPD having a total internal volume of 5 ml, wash volumes may range from 50 ml to 500 ml. Wash solutions are pH-buffered to neutral or mildly basic pH (i.e., pH 7.0 to pH 8.0) and contain salts (NaCl and/or others) at moderate concentrations that will displace biomolecules with weaker negative charge (such as serum albumins) from the anion-exchange filtration/adsorption medium, yet not displace captured EVs, VPs or VLPs (e.g., 200 mM to 400 mM NaCl). Wash buffers may also contain one or more non-ionic or zwitterionic surfactants such as Triton-X 100 or CHAPS to aid in solubilizing and removing lipids and lipoproteins from the anion-exchange filtration/adsorption medium, but at far lower concentrations than used in processing the SPD for recovery of total cell-free nucleic acids, such that EV membranes or viral envelopes will not be disrupted, e.g., Triton X-100, at less than 0.025% by volume.

Elution of captured EVs, VPs or VLPs from the SPD in partially purified and concentrated form is accomplished by passing relatively small volumes of an aqueous, high-salt elution buffer through the SPD by means of a syringe or pump. Elution volume is typically 2 to 10 device-volumes, e.g., 10 ml to 50 ml for an SPD having total internal volume of 5 ml. The elution buffer is adjusted to mildly basic pH (i.e., pH 7.4 to pH 8.0) and contains salt (NaCl and/or others) at a concentration high enough to displace bound EVs, VPs or VLPs from the anion-exchange filtration/adsorption medium so that they may be recovered from eluate exiting the SPD, e.g., 0.5 M to 0.9 NaCl for EVs, or 1 M to 2 M for VPs or VLPs, depending on the specific type(s) of particles to be recovered. Salt concentrations that elute EVs, VPs or VLPs may overlap the range of salt concentrations that elute non-vesicular cfNA. However, partial purifications of EVs, VPs or VLPs by differential elution will be possible in some instances, depending upon the type(s) of EVs, VPs or VLPs targeted for recovery.

EVs, VPs or VLPs recovered from the SPD by these methods will be enriched relative to their concentration in circulating plasma, but possibly still contaminated with non-vesicular cell-free nucleic acids, as the latter may not have been removed entirely by washing or differential elution steps. Further purification of EVs, VPs or VLPs away from non-vesicular cfNA and other plasma contaminants in the SPD eluate can be accomplished by methods such as density-gradient ultracentrifugation or size exclusion chromatography using, e.g., the Izon qEV system. The effectiveness of such methods will be improved if preceded by selective digestion of non-vesicular cfNA using DNases, RNases or a combination of DNases and RNases. Before being subjected to nuclease digestion or other purification steps, high-salt SPD eluates containing EVs, VPs or VLPs should be adjusted to near-physiological osmolarity in order to protect membrane integrity, through ultrafiltration or dialysis against a suitable replacement buffer such as phosphate-buffered saline (PBS).

As for total cell-free nucleic acids, yields of EVs, VPs or VLPs recovered from the SPD will vary widely among human subjects undergoing apheresis collection procedures and will depend upon the duration chosen for the collection procedure, i.e., the volume of plasma processed. For a maximum collection period of 2 to 3 hours, in which 50% to 75% of the circulating plasma will pass through the SPD, the total yield EVs recovered from one collection procedure by the methods outlined here may range up to 2-3×10¹³ or more.

Referring now to FIG. 6, an overview of methods for post-collection handling of the SPD to stabilize and preserve captured EVs, VPs or VLPs, and for subsequent laboratory processing to recover EVs, VPs or VLPs, in partially purified and concentrated form suitable for further downstream purification and analysis is illustrated according to one embodiment of the present invention with reference to the following steps. (1) removal from the apheresis procedure tubing set and stabilization; (2) packing for transport; (3) digestion of non-vesicular nucleic acids and/or proteins; (4) washing to remove impurities; (5) elution of EVs, VPs or VLPs; (6) de-salting and concentration; (7) yield and quality metrics. The steps produce an EV that is isolated from the EV-rich plasma and the EV isolated from the plasma at the SPD can be sent to a diagnostic genomics lab for further analysis.

One or more embodiments of the present invention utilize anion-exchange chromatography media to capture dilute nucleic acids, or extracellular vesicles (EVs), viral particles (VPs) or virus-like particles (VLPs) from biological fluids, most particularly circulating blood plasma. The claimed methods draw upon a large body of prior art in laboratory and industrial applications of anion-exchange chromatography. Relevant examples include (i) purification of plasmid or phage DNA from bacterial lysates; (ii) purification of cellular DNA or cfDNA from clinical specimens of blood or plasma drawn by syringe (but not from circulating plasma in the extracorporeal blood circuit of a patient undergoing apheresis, from which plasma may be reintroduced to the patient); (iii) removal of contaminating cfDNA shed from cultured mammalian host cells into the culture medium during biopharmaceutical production of recombinant proteins such as monoclonal antibodies; (iv) isolation and concentration of exosomes from cell lysates or culture supernatants, and (v) recovery and concentration of recombinant viral vectors from culture media of packaging cell lines.

However, the SPD and associated methods described herein are distinguished from prior applications of anion-exchange chromatography in that embodiments of the SPD described herein capture, concentrate and recover cfDNA, cfRNA, EVs, VPs or VLPs directly from a large portion of the total circulating plasma volume of a human subject (up to 2 liters or more of plasma), by working in conjunction with a clinical apheresis machine. This task entails very different challenges and physical constraints from in-vitro applications of anion-exchange chromatography to clinical lab specimens of body fluids after their collection by more conventional means, or to industrial volumes of cell culture medium. For example, cfDNA, cfRNA, EVs, VPs or VLPs to be captured must bind to an anion-exchange medium under physiological conditions, i.e., the circulating plasma must not be chemically altered in any way to facilitate binding if it is to be returned to the patient's body. Further, the SPD must be resistant to clogging by colloidal materials normally present in blood plasma, and permit high filtration flow rates, e.g., ≥50 ml/min, that can be sustained at the modest input pressures available from a clinical apheresis machine, e.g., ≤600 mmHg, yet fulfill these requirements with a small internal device volume that does not dangerously expand the total volume of the extracorporeal blood circuit during apheresis. Moreover, the SPD must be fully biocompatible and medically safe, i.e., not contain or release substances that might present hazards to the patient undergoing apheresis.

Note that in the specification and claims, “about” or “approximately” means within twenty percent (20%) of the numerical amount cited. All computer software disclosed herein may be embodied on any computer-readable medium (including combinations of mediums), including without limitation CD-ROMs, DVD-ROMs, hard drives (local or network storage device), USB keys, other removable drives, ROM, and firmware.

Although the invention has been described in detail with particular reference to these embodiments, other embodiments can achieve the same results. Variations and modifications of the present invention will be obvious to those skilled in the art and it is intended to cover in the appended claims all such modifications and equivalents.

For example, in one embodiment the SPD, the filtration/adsorption media is not itself a chemical entity that binds with affinity to a specific target such as a defined nucleic acid sequence or a defined amino acid sequence or protein structure, nor is the filtration/adsorption media itself conjugated to a chemical entity that binds with specific affinity to a defined nucleic acid sequence or a defined amino acid sequence or protein structure. Such a chemical entity may be for example an antibody, DNA sequence, RNA sequence, PRNA sequence or amino acid sequence. In one embodiment of the present invention, the filtration/adsorption medium does not comprise conjugated affinity ligands such as Histone H1 or other DNA/RNA binding proteins, anti-histone antibodies, anti-nucleosome antibodies, anti-DNA antibodies, DNA intercalating agents, DNA-binding polymers such as PEI or PAMAM, or lectins, as any of these would entail greater complexity, higher costs and obstacles to regulatory approvals.

In one embodiment of the SPD and method of using the SPD in an apheresis collection procedure, the volume of plasma from which cfNA or EVs, VPs or VLPs are captured is greater than volumes of 0.2 to 4 ml, for example, 500 ml to 1 liter or greater than 1 liter, for example 2 liters or more. In some embodiments the biological fluid, for example plasma, that is processed is reintroduced to the body during the apheresis procedure.

In one embodiment the SPD does not comprise an apheresis device or system, i.e., it has no function in separating blood plasma from formed elements of the blood such as RBCs, WBCs and platelets but instead, an embodiment of the SPD is an accessory filter to be used with existing clinical apheresis machines that have regulatory approvals in place, for example the Spectra OPTIA apheresis device (Terumo BCT, Golden CO).

In one embodiment, the SPD and methods described herein do not purify blood or blood plasma but instead embodiments of the SPD and methods recover cell-free nucleic acids, or intact EVs, VPs or VLPs directly from the blood circulation for analytical purposes. In one embodiment of the SPD and methods of use are not intended or proposed to produce sustained reductions in levels of cfDNA, cfRNA, EVs, VPs or VLPs present in the circulation of patients undergoing apheresis for collection of these substances via the SPD as diagnostic or prognostic biomarkers. Neither is use of the SPD intended or proposed to provide any direct therapeutic benefit to patients undergoing collection procedures.

The entire disclosures of all references, applications, patents, and publications cited above are hereby incorporated by reference.

1. A secondary plasma device (SPD) for use with extracorporeal blood processing systems having an extracorporeal blood tubing circuit wherein the SPD is capable of collecting circulating, cell-free nucleic acids (cfNA) directly from plasma separated from a subject's blood by the extracorporeal blood processing system, the SPD comprising:
a sterile capsule having:
an inlet port,
an outlet port, and
a filtration/adsorption medium positioned within the capsule a filtration/adsorption medium,
wherein the inlet port is configured to connect to the tubing circuit at a point distal to a hemoseparator and prior to a plasma collection reservoir or a treated plasma bag, in fluid communication with plasma previously separated from formed elements of subject's blood such that the plasma enters the SPD through the inlet port under pressure and contacts the filtration/adsorption medium prior to exiting through the outlet port,
wherein the outlet port is configured to connect to a return path portion of the tubing circuit leading back to the subject;
wherein the filtration/adsorption medium bears a fixed positive charge at physiological pH, thereby configured to function as an anion exchange substrate which captures cfNA from the previously-separated plasma passing through the SPD; and
wherein the filtration/adsorption medium is configured to collect cfNA in quantities of at least 10-fold more than an amount of cfNA recoverable from a 5 ml venipuncture blood draw specimen.

2. The SPD of claim 1 wherein the extracorporeal blood processing system comprises a system selected from the group consisting of a clinical apheresis system, a therapeutic apheresis system, a dedicated apheresis machine for plasma donation, a dedicated apheresis machine for platelet donation, a renal dialysis machine, a cardiopulmonary bypass systems, a heart-lung machine, and an extracorporeal membrane oxygenation machine.

3. The SPD of claim 1 wherein the positive charge is provided by chemical groups integrated to the filtration/adsorption medium.

4. The SPD of claim 1, wherein captured cfNA comprises one or more captured molecules selected from the group consisting of free molecules of DNA, free molecules of RNA, DNA complexed with other macromolecules, RNA complexed with other macromolecules, DNA enclosed within membrane-bound extracellular vesicles, RNA enclosed within membrane-bound extracellular vesicles, DNA in a virus particle, RNA in a virus particle, DNA in a virus-like particle, and RNA in a virus-like particle.

5. The SPD of claim 1 wherein the filtration/adsorption medium comprises one or more media selected from the group consisting of a bed of polymeric resin beads, a cast monolithic structure of mesoporous polymeric material, and a polymeric microfiltration membrane, wherein the one or more media are imbued with a high density of positively charged chemical groups in the course of their manufacture or through post-manufacture treatments.

6. The SPD of claim 1, wherein the filtration/adsorption medium comprises a bundle of hollow-fiber micro-filtration membranes composed of polymers selected from the group consisting of polysulfone, polyethersulfone, polyvinylidine fluoride, cellulose acetate, and copolymers of these materials with other constituents, formulated or modified to incorporate positively charged chemical groups such as protonated amines or imines.

7. The SPD of claim 1, wherein an internal volume of the SPD comprises 10 ml or less, thereby avoiding potentially unsafe additions to a total volume of the extracorporeal blood circuit and minimizing volumes required for washing and eluting the SPD.

8. The SPD of claim 1, wherein the SPD can attain plasma flow rates of about 10 to 50 ml/minute or greater at inlet pressures of about 50 to 600 mmHg.

9. The SPD of claim 1, wherein the filtration/adsorption medium and enclosing capsule comprise biocompatible materials suitable for use in a blood-contact medical device.

10. A method of capturing cell-free nucleic acids (cfNA) from the blood circulation by the SPD of claim 1 in a collection procedure using a clinical apheresis machine or similar extracorporeal blood-processing system, the method comprising:
connecting the inlet port and the outlet port of the SPD of claim 1 into the tubing circuit of an extracorporeal blood processing system at a point between a plasma pump and a plasma collection reservoir or a treated plasma bag and distal to a separator of blood cells and platelets from most of the plasma of the extracorporeal blood so that flow of plasma essentially free of formed elements is received under pressure into the SPD;
flowing the extracorporeal cell-free plasma through the SPD for a period of time;
collecting cfNA in the filtration/adsorption medium, while essentially all other plasma components pass through the SPD to exit to the plasma reservoir or the treated plasma bag for eventual return to the subject's bloodstream; and
stabilizing within the SPD the collected cfNA to protect from degradation prior to recovery of captured cfNA.

11. The method of claim 10 wherein the collected cfNA comprises one or more cfNA selected from the group consisting of free-floating molecules of cfNA, cfNA complexed with other biological macromolecules, cfNA contained within membrane-bound extracellular vesicles (EVs), cfNA in virus particles (VPs), and cfNA in virus-like particles (VLPs).

12. The method of claim 10 further comprising:
disconnecting the SPD from the tubing circuit immediately after the end of the cfNA collection procedure;
flushing the disconnected SPD immediately with several device-volumes of a preservative solution injected with a syringe through the inlet port of the device; and
sealing the inlet port and outlet port with water-tight caps that will remain securely fastened in place during transport and until the SPD is processed for recovery of the captured cfNA.

13. The method of claim 12, wherein the preservative solution used to stabilize captured cfNA in the SPD of claim 1 includes one or more chemical agents selected from the group consisting of citrate and EDTA that chelate divalent cations at concentrations sufficient to inhibit activity of nucleolytic enzymes present in plasma that might otherwise degrade the captured cfNA.

14. The method of claim 12, wherein the preservative solution may include one or more proteolytic enzymes at concentrations sufficient to digest nucleases present in plasma and other plasma proteins such as serum albumins regarded retained by the SPD as contaminants of the captured cfNA specimen.

15. The method of claim 12, wherein the preservative solution may include one or more non-ionic or zwitterionic surfactants at concentration sufficient to solubilize any contaminating plasma lipids or lipoprotein complexes retained by the SPD as contaminants of the captured cfNA specimen.

16. The method of claim 12, wherein the preservative solution is formulated with ionic strength and pH that allow the captured cfNA or captured extra-cellular vesicles (EVs), virus particles (VPs) and virus-like particles (VLPs) to remain bound on the filtration/adsorption medium.

17. (canceled)

18. A method for post-collection processing of an SPD of claim 1 to recover captured cfNA in a state of purity and concentration suitable for use with reverse transcription, polymerase chain reaction, DNA sequencing and other enzymatic or serological assays for other biochemical alterations, e.g., DNA damage or epigenetic modifications, said method comprising:
washing to remove digested or partially digested proteins, solubilized lipids and other contaminating plasma substances from the SPD of claim 1;
eluting of bound nucleic acids from the anion-exchange filtration/adsorption medium of the SPD of claim 1 with a small volume of a high-salt elution buffer; and
desalting and further concentrating of the eluted nucleic acids;
wherein post-collection processing of the SPD to recover captured cfNA in purified and concentrated form further comprises one or more washing steps in which 10 to 100 device volumes of a wash buffer are passed through the SPD by means of a syringe or pump, said wash buffer being adjusted to mildly basic pH and containing salt at moderate concentrations that will displace biomolecules with weaker negative charge from the anion-exchange filtration/adsorption medium yet not displace captured cfNA, and containing one or more non-ionic or zwitterionic surfactants at concentrations sufficient to solubilize lipids and lipoproteins and promote their removal from the filtration/adsorption medium.

19. A method for post-collection processing of an SPD of claim 1 to recover captured cfNA in a state of purity and concentration suitable for use with reverse transcription, polymerase chain reaction, DNA sequencing and other enzymatic or serological assays for other biochemical alterations, e.g., DNA damage or epigenetic modifications, said method comprising:
washing to remove digested or partially digested proteins, solubilized lipids and other contaminating plasma substances from the SPD of claim 1;
eluting of bound nucleic acids from the anion-exchange filtration/adsorption medium of the SPD of claim 1 with a small volume of a high-salt elution buffer; and
desalting and further concentrating of the eluted nucleic acids;
wherein post-collection processing of the SPD to recover captured cfNA in purified and concentrated form further comprises elution of cfNA by passing 2 to 10 device volumes of an aqueous, high-salt elution buffer through the SPD by means of a syringe or pump, said elution buffer being adjusted to mildly basic pH and containing salt at a concentration sufficient to displace bound cfNA from the anion-exchange filtration/adsorption medium so that they are dissolved in eluate exiting the SPD.

20. A method for post-collection processing of an SPD of claim 1 to recover captured cfNA in a state of purity and concentration suitable for use with reverse transcription, polymerase chain reaction, DNA sequencing and other enzymatic or serological assays for other biochemical alterations, e.g., DNA damage or epigenetic modifications, said method comprising:
washing to remove digested or partially digested proteins, solubilized lipids and other contaminating plasma substances from the SPD of claim 1;
eluting of bound nucleic acids from the anion-exchange filtration/adsorption medium of the SPD of claim 1 with a small volume of a high-salt elution buffer; and
desalting and further concentrating of the eluted nucleic acids;
wherein post-collection processing of the SPD to recover captured cfNA in purified and concentrated form further comprises desalting and final concentration of the eluted cfNA by standard means such as precipitation with alcohol, dialysis, ultrafiltration, or capture on nucleic acid-binding membranes or nucleic acid-binding paramagnetic beads.

21. A method for post-collection processing of the SPD of claim 1 to recover captured extra-cellular vesicles (EVs), virus particles (VPs) or virus-like particles (VLPs) in a state of purity and concentration suitable for use with one or more analyses selected from the group consisting of reverse transcription, polymerase chain reaction, DNA sequencing, enzymatic assays and serological assays, said method comprising:
washing to remove plasma proteins, plasma lipids and other contaminating plasma substances from the SPD of claim 1;
eluting bound EVs, VPs or VLPs from the anion-exchange filtration/adsorption medium of the SPD of claim 1 with a small volume of a high-salt elution buffer; and
desalting and further concentrating of the eluted EVs, VPs or VLPs.

22. The method of claim 21, wherein post-collection processing of the SPD to recover captured EVs, VPs or VLPs in partially purified and concentrated form further comprises one or more washing steps in which 10 to 100 device volumes of a wash buffer are passed through the SPD by means of a syringe or pump, said wash buffer being adjusted to mildly basic pH (i.e., pH7.4 to pH 8.0) and containing salt (NaCl and/or others) at moderate concentrations (e.g., 200 mM to 400 mM NaCl) that will not displace captured EVs, VPs or VLPs from the anion-exchange filtration/adsorption medium, but will displace biomolecules or colloidal complexes with weaker negative charge.

23. The method of claim 21, wherein post-collection processing of the SPD to recover captured EVs, VPs or VLPs in partially purified and concentrated form further comprises elution of EVs, VPs or VLPs by passing 2 to 10 device volumes of an aqueous, high-salt elution buffer through the SPD by means of a syringe or pump, said elution buffer being adjusted to mildly basic pH (i.e., pH 7.4 to pH 8.0) and containing salt (NaCl and/or others) at a concentration sufficient to displace bound EVs, VPs or VLPs from the anion-exchange filtration/adsorption medium so that they are resuspended in eluate exiting the SPD—e.g., 0.5 M to 0.9 NaCl for EVs, or 1 M to 2 M for VPs or VLPs.

24. The method of claim 21, wherein post-collection processing of the SPD to recover captured EVs, VPs or VLPs in partially purified and concentrated form further comprises adjusting high-salt SPD eluates containing EVs, VPs or VLPs to near-physiological osmolarity in order to protect membrane integrity, through standard methods such as dialysis against a suitable replacement buffer, e.g., phosphate-buffered saline (PBS), or ultrafiltration with washing and final resuspension in PBS.
